(19) United States
(12) Patent Application Publication
(10) Pub. No.: US 2015/0068533 A1
(43) Pub. Date: Mar. 12, 2015

(54) LOCAL COMPOSITION TO TREAT STRESS URINARY INCONTINENCE IN A FEMALE SUBJECT

(71) Applicant: Carlo GHISALBERTI, Sao Paulo - SP (BR)

(72) Inventor: Carlo Ghisalberti, Sao Paulo (BR)

(21) Appl. No.: 14/389,947

(22) PCT Filed: Mar. 28, 2013
(86) PCT No.: PCT/IB2013/000549

§ 371 Date: Oct. 1, 2014

(30) Foreign Application Priority Data
Apr. 2, 2012 (IT) MI2012A000536

(51) Int. Cl. A61K 31/132; A61M 31/00; A61F 6/04

(52) U.S. Cl. CPC A61K 31/132; A61F 6/04; A61M 31/00
USPC 128/844; 564/512; 514/674

(57) ABSTRACT

A composition for local application contains spermidine for the treatment of the stress urinary incontinence in a female subject. The composition is intended for transvaginal application to release spermidine onto external sphincter, urethra and endopelvic fascia, or for intravescial instillation. A variant composition takes the form of a condom lubricant comprising spermidine for the local release thereof during sexual intercourse, as an alternative or complementary to the local self-administration.

### FIELD OF THE INVENTION

The invention relates to topical compositions to treat stress urinary incontinence (SUI).

### BACKGROUND

Urinary incontinence is the involuntary loss of urine in inappropriate moment and place, a diffuse disturbance with quite high prevalence in the female gender. The major forms of urinary incontinence are clinically termed as follows:

from stress (SUI), i.e. associated to physical strain such as cough, sneeze, laughter, etc.; and

from urgency (UUI), i.e. characterized by a sudden compelling micturition stimulus.

Around 50% of the female incontinence is classified as SUI, a percentage that decreases with ageing when UUI become more frequent. The two forms may even coexist in the mixed urinary incontinence (MUI).

In women incontinence often shows up in conjunction to an altered pelvic condition with prolapses of organs like urethra (cystocele), rectus (rectocele), uterus (hysterocele) and/or part of the intestinal tract.

In the review of Davila G W (Adv Urol. 2011; 2011:176498; 1-14) a variety of available pharmacological and medical device treatments are compared and overviewed with focus on “pros & cons” in patients with significant urinary loss according to variables such as age and general condition, with particular attention to the risk of a surgical intervention.

Interestingly, SUI and MUI are often confused by clinicians and, consequently, mistreated. In this respect Petros P P P (Int Urogynecol J. 2011; 22:919-921) pointed out that: “a) SUI and UUI are different symptoms with often different anatomical causation and so should be treated separately; (b) It is illogical to group urgency with SUI. Urgency may also be associated with frequency, nocturia, abnormal emptying and pelvic pain in patients with no SUI (“posterior fornix syndrome”); and (c) there is growing evidence that urgency may be cured by surgical correction of a cystocele and/or apical prolapse in up to 80% of patients who do not have SUI.” Therefore, a therapeutic plan directed to female urinary incontinence shall be differentiated accordingly.

A survey on candidate drugs for SUI in a study disclosed fesoterodine (Pfizer, phase II) and duloxetine (Boheringer, phase III), both apparently in stand-by. The approved drugs with indication in SUI are solifenacine and tolterodine. These molecules are primarily conceived for UUI, hence bring about little, if any, medical utility in SUI.

Estrogens may be a potential aid. However, the correlation between hormonal status in pre- or post-menopause and urinary incontinence is highly controversial. Various authors did not find a link between the estradiol blood level and the pathology; whilst others encourage the use of estrogens in SUI. Unfortunately even low-dose estrogens increase the exposure to neoplastic risk, endometriosis, and unfavourable cardiovascular events.

Therefore it remains a large unmet need for novel therapeutic agents with high efficacy coupled with an excellent safety profile for the treatment of SUI.

### SUMMARY

It was surprisingly discovered that spermidine represents an effective treatment in subjects with stress urinary incontinence (SUI).

In an aspect, the invention refers to a local composition comprising spermidine as salt or a supramolecular complex thereof to treat SUI.

In another aspect, the invention refers to a local composition to treat SUI comprising spermidine from about 0.1% to 0.001% w/v of the composition.

In yet another aspect, the inventive composition is conceived for the local application with transvaginal action on external sphincter, urethra and endopelvic fascia.

In a next aspect, the inventive composition is conceived for intravescical instillation.

In a further aspect, the inventive composition is a lubricating fluid for a condom wherein spermidine is release during intercourse.

In a yet further aspect, spermidine is partially or fully substituted with spermine.

These and other features of the invention are best described below.

### BRIEF DESCRIPTION OF THE DRAWING

FIG. 1 is graph of self-assessment in a subject by three 4-week questionnaires (ICQ-7, UDI-6, VALQ-5; see Ex. 1 for meanings) with scores at baseline (0); after 4+4 weeks (W4 and W8); a period of suspension (W12-W20); and next 4 weeks of treatment (W24).

### DETAILED DESCRIPTION

The present invention refers to a medicinal, local composition comprising spermidine for the treatment of stress urinary incontinence (SUI) in a female subject.

The expression “stress urinary incontinence” as used herein is referred to loss of urine associated to physical strain and sudden moves such as in cough, sneeze, laughter, etc.

The active ingredient of the present invention is spermidine, a substance of structure NH₂(CH₂)₃NH(CH₂)₄NH₂, and formula C₇H₁₉N₃.

Spermidine may be easily incorporated as salt or complex in gynecologic compositions or as pure amine in water-free or emulsified gynecologic compositions.

Example of salts are addition salt formed by spermidine and inorganic acid such as HCl, H₂SO₄, H₃BO₃, H₃PO₄, etc., or with organic acid such as methylsulfonic acid, ascorbic acid, glycolic acid, lactic acid, pyruvic acid, citric acid, and the like.

If spermidine as free amine is used, a partial or full exchange may take place in situ in contact with acidic substances within the composition. Analogously, a simple salt such as spermidine 3HCl may undergo acid-base exchange within the composition medium.

In a further embodiment, the composition of invention comprises a supramolecular complex with spermidine for the sustained release thereof.

The expression “supramolecular complex” as used herein includes polyacid complex formed by polyanionic polymer(s) and spermidine, and inclusion complex of spermidine into cyclodextrins. Technical and operative details of such supramolecular complexes are enclosed in our co-pending applications WO12/017288 and WO12/017290.

The main inventive object is a topical medicinal composition for treating SUI, wherein “medicinal composition” include drugs, medical devices, and lenitive cosmetics intended for the mentioned medical need.

The inventive composition will comprise spermidine in concentration from about 1% to 0.0001% w/v, more preferably from about 0.1% to 0.001% w/v, even more preferably around 0.01% w/v of the composition.

Therefore, the unitary dose of spermidine will be comprised between 1 mmoles and 1 nmoles/unit dose, preferably between 100 μmoles e 10 nmoles/unit dose, even more preferably around 10 μmoles and 100 nmoles/unit dose.

The compositions according to the invention to be administered vaginally can be fluid or semi-fluid forms such as of gel, cream, ointment, solution, and the like, or in solid form such as tablets, capsule, pessaries, ovules, etc. for the release spermidine.

The trasvaginal composition according to the invention may also be solid preparations such as vaginal ovules, plugs, and tablets having a content of around 1 μmole of spermidine per unit dose.

Said compositions are suitable for treating SUI for example by the vaginal route. The expression “transvaginal application” or “vaginal route” or any combination thereof as used herein mean the application along the vulva envisaging the transvaginal absorption with consequent activity on external sphincter, urethra and endopelvic fascia.

In another embodiment the inventive composition are conceived for intravescical administration having a content of around 1 μmole of spermidine per unit dose.

The inventive composition may be packaged in ordinary Al or plastic tubes; or as spray, mousse and other means for the application onto external genitalia by direct contact with hand, or for application onto vagina by suitable devices.

Excipients and auxiliary active ingredients to produce a composition with proper patient compliance, acceptability to regulatory authorities, and cost control are known, e.g. Garg S et al. in Compendium of Pharmaceutical Excipients for Vaginal Formulations Pharmaceutical Technology, Drug Delivery 2001, 14-24, who listed several excipients with their functional classification, allowed concentrations, and regulatory status.

Transvaginal and intravescical routes may be aided by auxiliary devices (applicators, or dosers) which are known to the expert of the art. Indeed, these devices may further increase the therapeutic efficacy if compared to the plain application on the meatus area.

In a particular embodiment, the inventive composition is a lubricant for condom comprising spermidine in a therapeutically effective amount to elicit urogenital tissue repair during the common, thereto protected sexual activity.

Said condom lubricant will comprise from 100 to 0.1 μmoles of spermidine for each single condom, more preferably from 10 to 1 μmoles of spermidine. The regular use of such modified condoms will allow the local release of spermidine during intercourses for the benefit of the female partner complaining SUI. This can be a suitable alternative or complement the local self-administration of an inventive composition.

The present composition may include other additional active ingredients. Examples include, but not limited to, anti-infective agent such as estgenic substances, antibiotics, anti-fungals, antivirals, biocides; heparins; gabaergics; anti-inflammatories; immuno-suppressant; plant or algal extracts; antihistamines; antioxidants; as well as astringents, vitamins, deodorants, preservatives, and other customary ingredients.

In another embodiment, spermidine is fully or partially replaced by the PA spermine, a substance of structure NH₂(CH₂)₃NH(CH₂)₄NH(CH₂)₃NH₂ and formula C₁₀H₂₆N₄.

Other inventive compositions may similarly contain a variety of complementary ingredients as those skilled in the art can select using conventional criteria.

### EXAMPLES

### Example 1

### Test formulation

An investigational device (ID) was prepared with the ingredients as set forth below.

| Ingredient | amount per 100 ml | |
|---|---|---|
| Spermidine HCl | 10 | mg |
| Sodium hyaluronate | 2.0 | g |
| Sodium benzoate | 0.3 | g |
| Benzyl alcohol | 0.3 | g |
| Phenoxyethanol | 0.5 | g |

| | |
|---|---|
| Citric acid | q.b. to pH 5.35 |
| Purified water | q.b. to 100 ml |

The resulting transparent gel was packaged in 30 ml Al tube in a GMP lab facility.

### Example 2

### Pilot Study on SUI

Herein after is designed of a protocol for the clinical validation of SUI treatment with spermidine by transvaginal administration.

### Primary Endpoint: Efficacy Scores

Changes were recorded by 4-week requested questionnaire, namely: International Consultation on Incontinence Questionnaire (ICIQ-7 Short Form); Urogenital Distress Inventory (UDI-6 Short Form); Vaginal Atrophy and Libido Questionnaire (VALQ-5 Short Form), whose score are illustrated in Table I, II, and III, respectively.

### Secondary Endpoint: Tolerability and Safety

Tolerability, scored from excellent to poor, is included on patient's questionnaire. Safety was monitored from the adverse events (AE) report, that were recorded at all visits from subject experience, to be reported and described in full length with special reference to manifestation, intensity and duration.

### Inclusion Criteria

Main inclusion criteria consist of:

signature of the informed consensus;

female subjects complaining the typical SUI burdens;

collaborative subjects able to follow instruction and protocol schedule.

### Exclusion Criteria

subjects unable to supply a valid informed consensus;

female subjects with evident manifestations of UUI or MUI;

patient with a history of trauma, local surgery, or neurodegenerative pathologies (e.g., multiple sclerosis) that potentially alter the conditions of nerves in the lower urinary tract;

patient with a history of faecal incontinence;

patient with a history of continuous and unconscious loss of urine;

corrective surgery to correct SUI or uterus prolapse within the 6 previous months;

rehabilitative therapy of urethra or electro-stimulation within the 2 weeks proceeding the first visit; or if the patient were planning to undergo;

patient with active or recurrent (means >6 episodes per year) infection on urethral tract as assessed by clinical history, clinical symptoms, or laboratory means.

### General Design of the Study

Visit 1 (0): enrolment with baseline assessment, ID delivery with instruction to start treatment by 3 doses per week by manual applications on the vulvar tract.

Visit 2 (week 4, W4): after 4 weeks of treatment, general assessment and next ID delivery for a new cycle with indication of 2 doses per week by the above route.

Visit 3 (week 8, W8): after the next 4 weeks of treatment, general assessment and questionnaires review.

Follow-up by phone calls during 12 weeks (W12-W20) of rest (without treatment). In case of only partial remission, a treatment prosecution is conceived.

Visit 4 (week 20, W20): ID delivered along with a set of vaginal applicator of 2.5 mL, instruction to start treatment by 3 doses per week by internal application onto vagina.

Visit 5 (week 24, W24): end of treatment with delivery of the filled questionnaires.

TABLE I
ICQ-7 (International Consultation on
Incontinence Questionnaire) score

| | | |
|---|---|---|
| 3. How often do you leack | never | 0 |
| urine? | about once a week or less | 1 |
| | often | |
| | two or three times a week | 2 |
| | about once a day | 3 |
| | several times a day | 4 |
| | all the time | 5 |
| 4. How much urine do you | none | 0 |
| usually leak (whether you | a small amount | 2 |
| wear protection or not)? | a moderate amount | 4 |
| | a large amount | 6 |
| 5. Overall, how much does | Please ring a number between | 0 |
| leaking urine interfere with | 0 (not at all) and 10 (a great | 1 |
| your everyday life? | deal) | 2 |
| | | 3 |
| | | 4 |
| | | 5 |
| | | 6 |
| | | 7 |
| | | 8 |
| | | 9 |
| | | 10 |

NB Other questionnaire points (1, 2, 6, and 7) relate the patient information.

TABLE II
UDI-6 (Urogenital Distress Inventory) score

| Has urine leakage affected | Not at | Sligh- | Moder- | |
|---|---|---|---|---|
| your . . . | all | tly | ately | Greatly |
| 1. Ability to do household chores | 0 | 1 | 2 | 3 |
| (cooking, housecleaning, laundry)? | | | | |
| 2. Physical recreation such as | 0 | 1 | 2 | 3 |
| walking, swimming, or other | | | | |
| exercise? | | | | |
| 3. Entertainment activities (movies, | 0 | 1 | 2 | 3 |
| concerts, etc.)? | | | | |
| 4. Ability to travel by car or bus | 0 | 1 | 2 | 3 |
| more than 30 minutes from home? | | | | |
| 5. Participation in social activities | 0 | 1 | 2 | 3 |
| outside your home? | | | | |
| 6. Emotional health (nervousness, | 0 | 1 | 2 | 3 |
| depression, etc.)? | | | | |
| 7. Feeling frustrated? | 0 | 1 | 2 | 3 |

TABLE III
VALQ-5 (Vaginal Atrophy and Libido Questionnaire) score

| How do you perceive your | | | | |
|---|---|---|---|---|
| vulvovaginal condition? | None | Light | Moderate | Severe |
| Vaginal itching | 0 | 1 | 2 | 3 |
| Vaginal burning | 0 | 1 | 2 | 3 |
| Vaginal dryness | 0 | 1 | 2 | 3 |
| Dyspareunia | 0 | 1 | 2 | 3 |
| During the treatment, do | None | Light | Moderate | Strong |
| you perceive any change in | | | | |
| libido? (expresses as) | | | | |
| Desire of sexual | 3 | 2 | 1 | 0 |
| intercourses | | | | |

The three questionnaires serve to monitor the uro-genital burdens associated with SUI by the assessment of urine loss, episodes and discomfort intensity, as well as an eventual vaginal issue.

### Doses and Treatment Regimen

IP was delivered at visit 1 and visit 2 with instruction to apply on vulvar area by hand 3 times per week during the first 4 weeks; followed by 2 times per week during the next 4 weeks, respectively.

In case of partial remission, the protocol could be extended to further 4 weeks treatment. In this frame, IP was then delivered with 2.5 mL applicator for in-deep administration onto vagina with posology of 3 times per week for 4 weeks.

### Example 3

### Case Report

A Caucasian woman aged 42 years was asking for remediation of SUI that surged after pregnancy and childbirth. Health condition was generally good, beside some intolerances to dairy or fermented foodstuff. She was primiparous with regular menstrual cycles.

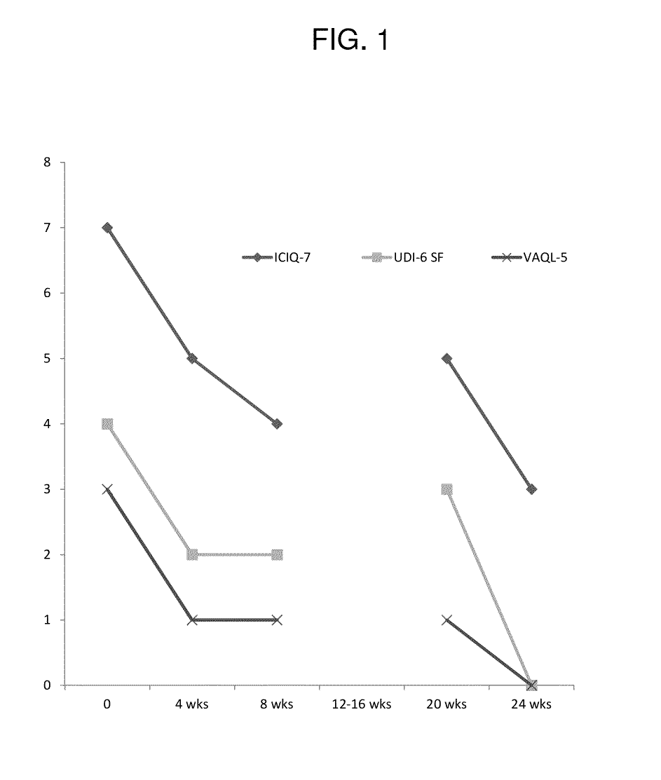

The subject entered the protocol with informed consensus. During the treatment she filled the questionnaires, by which remission resulted attenuated but not cancelled by the first 4-4 weeks of treatment. After a rest period, she underwent a 3th cycle of treatment with vaginal applicator. The questionnaires scores are highlighted in FIG. 1.

The tolerability was scored as excellent, with a

### Example 4

### Lubricating Fluid for Condoms

A transparent lube was prepared with the ingredients as set forth below.

| Ingredients | amount per 100 ml | |
|---|---|---|
| Spermidine 3HCl | 250 | mg |
| Propylene glycol | 14.9 | g |
| Polyethylene glycol 400 | 45.0 | g |
| Lubrajel ™ CG | 25.0 | g |

Lubrajel™ CG (Guardian Chem. Corp. Hauppauge, N.Y., USA) contains glycerine, glyceryl acrylate/acrylic acid copolymer, propylene glycol, parabens for preservation, and water in an amount of between about 24% to 34%, and said water being generally in a bound state, unable to undergo substantial reaction with the glycols present. Lubrajel is warmed at 40° C. and then added with macrogol, propylene glycol and spermidine.

The lubricant described in Table 1 may be applied to a condom in a conventional fashion, wherein a portion is shot into the interior of the condom and a portion is placed on the exterior. The so-coated condom may then be foil-wrapped using procedures common to the art. For coating purposes, it is preferred that 0.45 g to 0.75 g. of lubricant be applied to the exterior surface of the condom and 0.15 g to 0.45 g of the lubricant be applied to the interior surface. A typical method of applying lubricant to condom surfaces, such as is described in US 2002/0103414 A1, may be utilized in connection with the present invention to apply the curative lubricant on condom surfaces.

The above manipulation end up with a “functionalized condom” that is endowed of a local release onto the female urogenital tract of approximately 5 μmoles of spermidine during a single sexual intercourse.

The regular use of such condoms by male partner shall be encouraged by the female partner whenever she undergoes a chronic urinary incontinence of stress-type, e.g. a multiparous woman whose multiple pregnancies and childbirth had affected pelvic fascia and/or urethra status.

Another exemplary subject is a sexual active, elderly woman complaining for SUI, this being caused by the hormonal decay after the menopausal start.

Noteworthy, the use of functionalized condoms with a lubricating fluid such as illustrated in Example 4 can be performed as alternative, or in conjunction or sequentially (before or after) the self-administered dosage form comprising spermidine with the modality illustrated in Examples 1-3.

### Example 5

### Spermine Composition

The lube with ingredients as listed in Example 4 is conceived with an equivalent amount of spermine 4HCl in lieu of spermidine 3HCl.

It should be understood that the foregoing relates only to preferred embodiments and to applicative examples of the present invention and that numerous modifications or alterations may be made therein without departing from the spirit and the scope of the invention as set forth in the appended claims.

1. A medicinal composition comprising spermidine in a therapeutic effective amount for the treatment of stress urinary incontinence in a female subject.

2. The medicinal composition according to claim 1, wherein spermidine is present in an amount of from 100 μmoles to 10 nmoles per unit dose.

3. The medicinal composition according to claim 2, wherein spermidine is present in an amount of from 10 μmoles to 100 nmoles per unit dose.

4. The medicinal composition according to claim 1 in a form of a gel, a cream, an ovule, a solution, or a tablet, for transvaginal administration.

5. The medicinal composition according to claim 1 in a form of a solution for intravesical instillation.

6. The medicinal composition according to claim 4 provided with an applicator to aid in-deep self-administration.

7. A medicinal composition in a form of a lubricant for a condom comprising spermidine in a therapeutically effective amount to treat stress urinary incontinence in a female subject when the condom is employed in conjunction with the sexual activity.

8. The medicinal composition in the form of a lubricant for a condom according to claim 7, comprising spermidine in amount from 100 μmoles to 10 nmoles per unit dose.

9. The medicinal composition according to claim 1 further comprising spermine.

10. The medicinal composition according to claim 2 in a form of a gel, a cream, an ovule, a solution, or a tablet, for transvaginal administration.

11. The medicinal composition according to claim 3 in a form of a gel, a cream, an ovule, a solution, or a tablet, for transvaginal administration.

12. The medicinal composition according to claim 2 in a form of a solution for intravesical instillation.

13. The medicinal composition according to claim 3 in a form of a solution for intravesical instillation.

14. The medicinal composition according to claim 5 provided with an applicator to aid in-deep self-administration.

15. The medicinal composition in the form of a lubricant for a condom according to claim 7, wherein spermidine 3HCl is present at a concentration of about 250 mg per 100 ml of the lubricant.

16. The medicinal composition in the form of a lubricant for a condom according to claim 7 wherein the therapeutically effective amount of spermidine in the lubricant on a condom comprises from 100 to 0.1 μmoles of spermidine.

17. The medicinal composition in the form of a lubricant for a condom according to claim 7 comprising spermidine in a quantity that provides a local release onto the female urogenital tract of approximately 5 μmoles of spermidine during a single sexual intercourse.

18. A medicinal composition comprising spermine in a therapeutic effective amount for the treatment of stress urinary incontinence in a female subject.

19. A condom comprising a medicinal composition, in a form of a lubricant, comprising spermidine in therapeutically effective amount to treat stress urinary incontinence in a female subject when the condom is employed in conjunction with the sexual activity, wherein the lubricant on said condom comprises from 100 to 0.1 μmoles of spermidine.

20. A condom comprising a medicinal composition, in a form of a lubricant, comprising spermidine in therapeutically effective amount to treat stress urinary incontinence in a female subject when the condom is employed in conjunction with sexual activity, wherein said condom provides a local release onto a female's urogenital tract of about 5 μmoles of spermidine during a single act of sexual intercourse.
